(19) United States
(12) Patent Application Publication
(10) Pub. No.: US 2026/0314950 A1
(43) Pub. Date: Oct. 15, 2026

(54) SYSTEMS AND METHODS FOR ENDOSCOPIC SHAFT SHAPE SENSING

(71) Applicant: Noah Medical Corporation, San Carlos, CA (US)

(72) Inventors: Michael Ian LARKIN, Santa Barbara, CA (US); Enrique ROMO, Danville, CA (US)

(21) Appl. No.: 19/696,716

(22) Filed: Jun. 3, 2026

Related U.S. Application Data
(63) Continuation of application No. PCT/US2024/060028, filed on Dec. 13, 2024.
(60) Provisional application No. 63/615,545, filed on Dec. 28, 2023.

(51) Int. Cl. A61B 5/06; A61B 1/00; A61B 1/005; A61B 34/00; A61B 34/20; A61B 34/30; A61M 25/01

(52) U.S. Cl. CPC A61B 5/067; A61B 1/00057; A61B 1/0057; A61B 5/062; A61B 34/71; A61B 2034/2061; A61B 2034/301; A61M 2025/0166

(57) ABSTRACT

A method is provided for method for estimating shape of an elongated member of an articulating flexible endoscope. The method comprising: receiving sensor data acquired by a series of sensor pairs located at multiple points along a length of the elongated member, where the series of sensor pairs are mounted to a structure movable relative to the elongated member; calculating orientations of the elongated member at the multiple points; and estimating a shape of the elongated member based at least in part on the orientations.

### CROSS-REFERENCE

This application is a Continuation of International Application No. PCT/US2024/060028, filed on Dec. 13, 2024, which claims priority to U.S. Provisional Patent Application No. 63/615,545, filed on Dec. 28, 2023, which is entirely incorporated herein by reference.

### BACKGROUND

Endoscopy procedures use an endoscope to examine the interior of a hollow organ or cavity of the body. Unlike many other medical imaging techniques, endoscopes are inserted into the organ directly. Flexible endoscope that can deliver instinctive steering and control is useful in diagnosing and treating diseases that are accessible through any natural orifice in the body. Depending on the clinical indication, the endoscope may be designated as bronchoscope, ureteroscope, colonoscope, gastroscope, ENT scope, and various others. For example, flexible bronchoscope may be used for lung cancer diagnosis and/or surgical treatment. However, one challenge in bronchoscopy is reaching the upper lobe of the lung while navigating through the airways. In another example, flexible endoscopy has been used to inspect and treat disorders of the gastrointestinal (GI) tract without the need for creating an opening on the patient's body. The endoscope is introduced via the mouth or anus into the upper or lower GI tracts respectively.

Continuum robots and endoscopes are usually long and flexible. For example, endoscopes have long flexible shafts that are pushed or pulled through physiology to access target locations within the body. Endoscopes are designed to be flexible, allowing them to conform to the shape of various lumens within the body, such as airways in the lungs, blood vessels, the GI tract, bile passageway, urethra, and the like. However, due to this flexibility it is often difficult to know the shape of the endoscope or exactly where the endoscope is located within the body.

There are advantages of monitoring and/or knowing a three-dimensional shape of the endoscope (in whole or in part). For instance, knowledge of where the endoscope is within the body, and knowledge regarding the shape of the physiology traversed by the endoscope may be used to navigate the endoscope traversing the body. In another example, when the endoscope is within the colon, and the shape of the endoscope is known, a physician performing a colonoscopy can easily know if the colon is straight or is twisted and kinked, which is valuable information to have during a procedure.

Current devices and methods for measuring shape of a flexible endoscope are based on optical shape sensing techniques utilizing fiber optics with fiber Bragg gratings along the lengths of the fibers. However, such methods and devices are complex, requiring high-cost capital equipment for use, and the fiber optic shape sensing structures are expensive, making them inappropriate for single-use or disposable endoscope applications. Endoscopes are traditionally made to be re-usable, which may require thorough cleaning, dis-infection, and/or sterilization after each procedure. In most cases, cleaning, dis-infection, and sterilization may be aggressive processes to kill germs and/or bacteria. Such procedures may also be harsh on the endoscopes themselves. In contrast, single-use, disposable endoscopic devices can be packaged in sterile wrappers to avoid the risk of pathogenic cross-contamination of diseases such as HIV, hepatitis, and other pathogens. Hospitals generally welcome the convenience of single-use disposable products because they no longer have to be concerned with product age, overuse, breakage, malfunction, and sterilization.

### SUMMARY

Recognized herein is a need for a robotic endoscope that allows for measuring shape of a shaft portion of an endoscope in real-time with cost-efficiency. Recognized also herein are devices and systems comprising endoscopes which may be disposable and may not require extensive cleaning procedures. The present disclosure provides low-cost, single-use articulatable endoscope for diagnosis and treatment in various applications such as bronchoscopy, urology, gynecology, arthroscopy, orthopedics, ENT, gastro-intestine endoscopy, neurosurgery, colonoscopy, and various others. In some embodiments, the present disclosure provides a single-use, disposable, robotically controlled bronchoscope for use with a robotic system to enable diagnostic evaluation of lesions anywhere in the pulmonary anatomy. It should be noted that the provided endoscope systems can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

It should be noted that the provided modular endoscope components and various components of the device can be used in various minimally invasive surgical procedures, therapeutic or diagnostic procedures that involve various types of tissue including heart, bladder and lung tissue, and in other anatomical regions of a patient's body such as a digestive system, including but not limited to the esophagus, liver, stomach, colon, urinary tract, or a respiratory system, including but not limited to the bronchus, the lung, and various others.

In an aspect, a method for estimating shape of an elongated member of an articulable flexible endoscope is provided. The method comprises: receiving sensor data acquired by a series of sensor pairs located at multiple points along a length of the elongated member, where the series of sensor pairs are mounted to a structure movable relative to the elongated member; calculating orientations of the elongated member at the multiple points; and estimating a shape of the elongated member based at least in part on the orientations.

In a related yet separate aspect, a non-transitory computer-readable storage medium including instructions that, when executed by one or more processors, cause the one or more processors to perform operations comprising: receiving sensor data acquired by a series of sensor pairs located at multiple points along a length of the elongated member, where the series of sensor pairs are mounted to a structure movable relative to the elongated member; calculating orientations of the elongated member at the multiple points; and estimating a shape of the elongated member based at least in part on the orientations.

In some embodiments, each of the series of the sensor pairs comprise an accelerometer and a magnetometer. In some embodiments, the structure is actuated to have a translational motion relative to the elongated member to increase a density of the multiple points. In some cases, an impact of the translational motion is corrected from the orientations. For example, the impact of the translation motion comprises an acceleration of the translational motion. In some cases, the structures is actuated to move at a constant velocity. In some cases, the structure is a slidable cable and wherein the series of sensor pairs are affixed to the slidable cable. In some instances, the slidable cable is driven by an actuator located at an instrument driving mechanism removably coupled to the articulable flexible endoscope.

In some embodiments, the shape of the elongated member is estimated using linear approximation, circle section approximation, or cubic spline approximation. In some cases, the shape of the elongated member is estimated using a combination of at least two of linear approximation, circle section approximation, or cubic spline approximation based at least in part on a spacing of the series of sensor pairs. In some embodiments, the shape of the elongated member is estimated using a machine learning algorithm trained model. In some cases, the model is automatically updated based at least in part on feedback data.

In some embodiments, the method further comprises calibrating the series of sensor pairs based on a first known shape of the elongated member and a first corresponding series of orientations, and a second known shape of the elongated member and a second corresponding series of orientations. In some cases, the first known shape is a straight configuration and wherein the second shape is an arc shape. In some cases, the method further comprises storing a calibration data in a memory device incorporated into the elongated member.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example of combining data from a pair of an accelerometer and a magnetometer.

FIG. 2 schematically illustrates an endoscope with bends and sensors positioned along the length of the endoscope.

FIG. 3 shows methods of estimating a shape of the shaft.

FIG. 4 shows a specific shape used for estimating system performance via simulation.

FIG. 5 shows an example of the output of the simulation using the circle segment approximation.

FIG. 6 shows error in location as a function of sensor spacing for shafts with three different radii of curvatures.

FIG. 7 shows the error in location as a function of sensor spacing divide by the avg radius of curvature.

FIG. 8 shows error in location as a function of standard deviation in the measurement of the orientation angle for each sensor.

FIG. 9 shows error in location as a function of standard deviation in the spacing between sensors along the shaft.

FIGS. 10-12 show performance of the cubic splines-based estimation method.

FIGS. 13-15 show the simulated system performance as a function of the standard deviation in the measurement angle, the standard deviation in the spacing between sensors along the shaft, and the sensor spacing.

FIG. 16 illustrates an example of a flexible endoscope.

FIGS. 17-18 show an example of a disposable endoscope removably coupled to an IDM.

FIG. 19 show an example of a disposable endoscope removably coupled to an IDM.

FIG. 20 shows an example of a distal tip of an endoscope.

FIGS. 21 and 22 show examples of distal portion of the catheter with integrated imaging device and the illumination device.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

While exemplary embodiments will be primarily directed at a device or system for bronchoscopy and colonoscope, one of skill in the art will appreciate that this is not intended to be limiting, and the devices described herein may be used for other therapeutic or diagnostic procedures and in various anatomical regions of a patient's body. The provided device or system can be utilized in urology, gynecology, rhinology, otology, laryngoscopy, gastroenterology with the endoscopes, combined devices including endoscope and instruments, endoscopes with localization functions, one of skill in the art will appreciate that this is not intended to be limiting, and the devices described herein may be used for other therapeutic or diagnostic procedures and in other anatomical regions of a patient's body, such as such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat, and various others, in the forms of: NeuroendoScope, EncephaloScope, OphthalmoScope, OtoScope, RhinoScope, LaryngoScope, GastroScope, EsophagoScope, BronchoScope, ThoracoScope, PleuroScope, AngioScope, MediastinoScope, NephroScope, GastroScope, DuodenoScope, CholeodoScope, CholangioScope, LaparoScope, AmioScope, UreteroScope, HysteroScope, CystoScope, ProctoScope, ColonoScope, ArthroScope, SialendoScope, Orthopedic Endoscopes, and others, in combination with various tools or instruments.

The systems and apparatuses herein can be combined in one or more of many ways to provide improved diagnosis and therapy to a patient. Systems and apparatuses provided herein can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms distal and proximal may generally refer to locations referenced from the apparatus, and can be opposite of anatomical references. For example, a distal location of a primary shaft or catheter may correspond to a proximal location of an elongate member of the patient, and a proximal location of the primary sheath or catheter may correspond to a distal location of the elongate member of the patient.

### Endoscopic Shaft Shape Sensing Using Sequential Orientation Sensors

The present disclosure provides systems and methods for measuring the shape of at least a portion of an endoscope in real time. The shape sensing system may have a simplified design with low capital equipment costs. The methods and systems herein may allow for low per-procedure unit costs applicable to single-use devices.

Unlike the conventional fiber-optic based sensor that measures the shape directly, the shape sensing mechanism of the present disclosure may estimate a shape of a flexible shaft based on a series of orientations measured along a length of a flexible shaft. In some embodiments, the shape sensing device and mechanism herein may measure the orientation of a flexible shaft of an endoscope at multiple points along the length of the shaft. The orientation measured the multiple points along the length of the shaft may be with respect to the earth reference frame and the series of measured orientations may be combined to estimate a shape of the shaft.

In some embodiments, a series of orientation sensors may be disposed along a length of at least a portion of the shaft for measuring the orientation at multiple points. In some embodiments, to measure the orientation of the shaft with respect to the earth frame at multiple points along the length of the shaft, a series of inertial sensors may be placed at multiple points along a length of the shaft. In some embodiments, an inertial sensor may comprise a pair of an accelerometer and a magnetometer. For example, a series of micro-electro-mechanical system (MEMS) accelerometer and magnetometer sensor pairs may be placed along the length of the shaft.

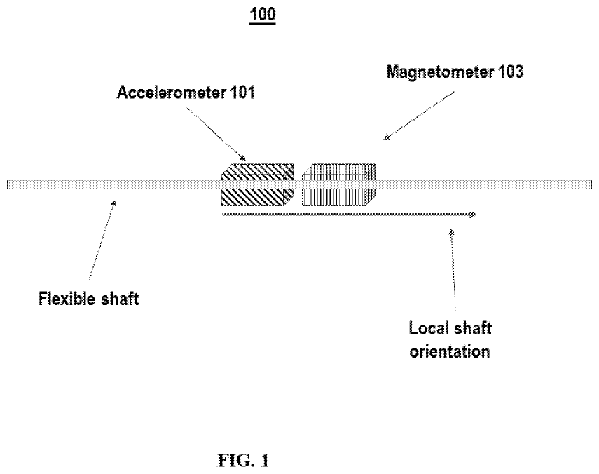

The method and system herein may combine the sensory data from a pair of accelerometer and magnetometer sensors to calculate an absolute orientation of the sensor pair with respect to the earth. In some cases, the orientation may be based on the accelerometer measuring the direction of gravity providing two degrees of freedom of orientation, and the magnetometer measuring the direction of the earth's magnetic field (or any local static field), providing two degrees of freedom of orientation. FIG. 1 schematically illustrates an example 100 of combining data from a pair of an accelerometer 101 and a magnetometer 103 allowing for the absolute orientation of the sensor with respect to the earth to be measured. By placing multiple pairs of the sensor along the shaft, changes in the orientation at locations along the shaft of an endoscope can be obtained.

In some cases, the accelerometer may be a two-axis or three-axis accelerometer. The accelerometer is able to measure an orientation of the sensor in an earth gravitational field. The orientation angle with respect to the earth/world gravitational field can be obtained by a rotation matrix from the ground reference frame to the accelerometer sensor body frame. The measurement may be based on a measure of a difference between an acceleration vector in the sensor's body frame and the gravitational vector. In some cases, the readout data of the accelerometer a may be based on the assumption that the sensor is experiencing a gravitational acceleration g. The gravitational acceleration can be obtained from the paired magnetometer which measures the Earth's magnetic field.

Combining the data from the pair of sensors allows the absolute orientation of the sensor pair to be known typically to less than ~1 deg. An inertial sensor may be used herein to refer to a motion sensor (e.g., a velocity sensor, an acceleration sensor such as an accelerometer), an orientation sensor (e.g., a gyroscope, inclinometer), or an IMU having one or more integrated motion sensors and/or one or more integrated orientation sensors. In some cases, other inertial sensors (motion or orientation sensors) may be utilized. For example, MEMS inertial measurement units (IMUs) typically incorporate an accelerometer, a gyroscope, and sometimes a magnetometer within a single integrated circuit (IC) package. While gyroscopic data is not required for this invention, in some embodiments gyroscopic data may be incorporated. Incorporating gyroscopic information can allow the system to know if the system is actively spinning, which can change the way in which the data from the accelerometer is being used.

An inertial sensor may provide sensing data relative to a single axis of motion. The axis of motion may correspond to an axis of the inertial sensor (e.g., a longitudinal axis). A plurality of inertial sensors can be used, with each inertial sensor providing measurements along a different axis of motion. For example, three angular accelerometers can be used to provide angular acceleration data along three different axes of motion. The three directions of motion may be orthogonal axes. One or more of the angular accelerometers may be configured to measure acceleration around a rotational axis. As another example, three gyroscopes can be used to provide orientation data about three different axes of rotation. The three axes of rotation may be orthogonal axes (e.g., roll axis, pitch axis, yaw axis). Alternatively, at least some or all of the inertial sensors may provide measurement relative to the same axes of motion. Such redundancy may be implemented, for instance, to improve measurement accuracy. Optionally, a single inertial sensor may be capable of providing sensing data relative to a plurality of axes. For example, an IMU including a plurality of accelerometers, magnetometers and gyroscopes can be used to generate acceleration data and orientation data with respect to up to six axes of motion.

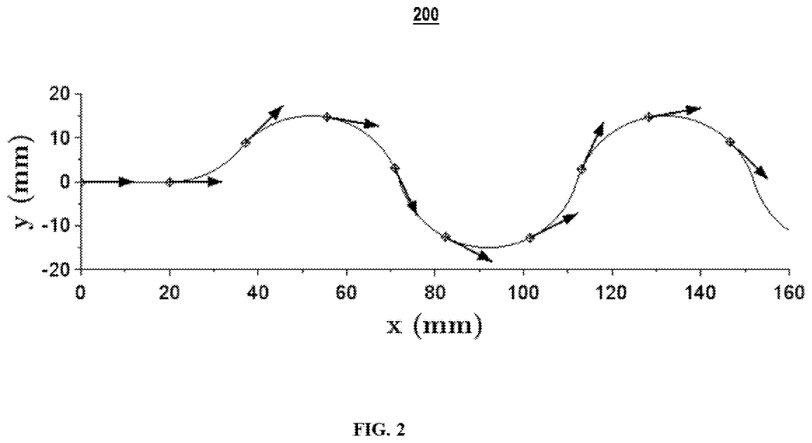

FIG. 2 schematically illustrates an endoscope 200 with bends and sensors positioned along the length of the endoscope. The sensor data points may indicate the orientation of the endoscope at the point at which they are mounted to. With a series of sensor pairs placed along a length of the shaft, the orientation of multiple points along the shaft can be calculated. An endoscopic shaft is not infinitely flexible, there is a minimum bend radius associated with a shaft. When the sensor pairs are spaced closely enough along a shaft, and if from those sensor pairs the orientation of the shaft is known at those two adjacent measurement points, then the shape of the shaft between those point can be accurately estimated. A shortened spacing of the sensor pairs along the length of the shaft may increase the accuracy of the estimation limited by the minimum bend radius of the shaft. The term “instrumented shaft” as utilized herein refers to a shaft with a series of sensor pairs placed at known positions along the length of the shaft.

Based on the local orientation at various points along the shaft measured by the series of sensor pairs, the derivative of the shaft at a series of points along the shaft is obtained. The method may estimate the shape of the shaft based on a 3D derivative along the shaft and calculating the 3D position along the shaft.

### Linear Approximation for Estimating Shape

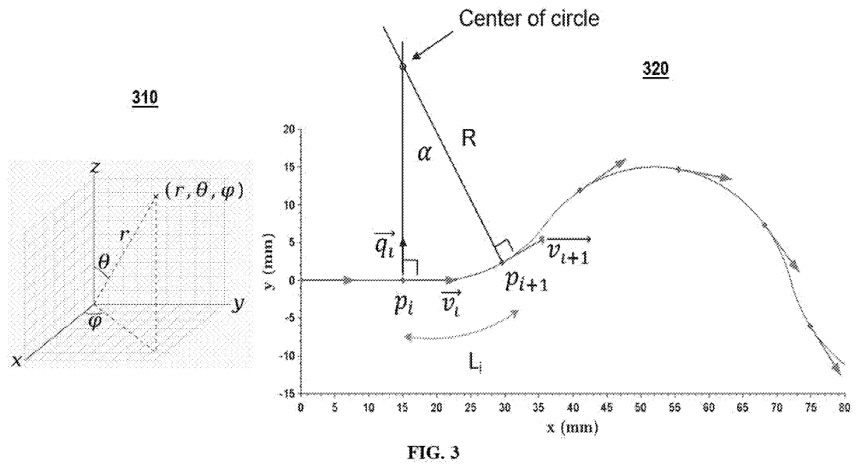

FIG. 3 shows methods of estimating a shape of the shaft. In some embodiments, the method to calculate the shape of the shaft from sequential orientations or derivative information may comprise linear approximation. The method may comprise summing all the individual slopes times the spacing between sensors. The method may be based on the assumption that the sections between sensors are linear. Thus, a denser placement of sensors may increase the accuracy for estimating the shape of the shaft.

Using the angle conventions 310 as shown in FIG. 3, the orientation of each sensor pair at sequential positions i along the shaft may be given as (θᵢ, φᵢ). With a chord length Li along the shaft between sequential sensor pairs, using the linear approximation the (Δxᵢ, Δyᵢ, ΔZᵢ) distance between segment i and i+1 is estimated as:

Δxᵢ=Lᵢ sin(θᵢ)cos(φᵢ),Δyᵢ=Lᵢ sin(θᵢ)sin(φᵢ),Δzᵢ=Lᵢ cos(θᵢ)

3D position of each sensor along the shaft, and so the shape of the shaft itself, may then be estimated from the sum of the delta distances as:

$${p}_{i} = ( {x}_{i} , {y}_{i} , {z}_{i} ) = ( {x}_{0} , {y}_{0} , {z}_{0} ) + ( {\sum }_{n = 1}^{i} Δ {x}_{n} , {\sum }_{n = 1}^{i} Δ {y}_{n} , {\sum }_{n = 1}^{i} Δ {z}_{n} )$$

With the position of the start of the sensing region (x₀, y₀, z₀) taken as a known starting location.

### Circle Segment Approximation for Estimating Shape

FIG. 3 shows another method 320 of estimating a shape of the shaft based on circle segment approximation. Instead of a linear approximation, the method 320 may calculate the shape of the shaft from sequential orientation or derivative information based on the assumption that the shaft segments between sensors can be approximated as sections of a circle. This is a variation of a quadratic approximation. This “circle segment” method 320 provides sufficient approximation accuracy with larger spacing between sensors than is required for the linear approximation. The method enforces the condition that the estimated position of each shaft segment meets at each sensor location, and also enforces the condition that the first derivatives of the estimated shape of the shaft are identical where the shaft segments meet.

The circle segment approximation method to use the measured derivative of locations along the shaft to estimate the location of the shaft is as follows:

Assume a starting location in three dimensions \vec{pₒ}=(x₀, y₀, z₀)

Use the measured orientation vectors \vec{vₗ} and \vec{vₗ₊₁} at two sequential points, as shown in FIG. 3 320, and the spacing Lᵢ between the points to calculate the radius R of a circle on which they would both be tangential, as:

$$\begin{matrix}\vec{{v}_{ι}} \cdot \vec{{v}_{ι + 1}} = {v}_{i} \vert \vert {v}_{i + 1} \vert cos ( {α}_{i} ) , \\ so {α}_{i} . = a cos ( \frac{\vec{{v}_{ι}} \cdot \vec{{v}_{ι + 1}}}{\vert {v}_{i} \vert \vert {v}_{i + 1} \vert} ) , and \\ {R}_{i} = \frac{{L}_{i}}{{α}_{i}}\end{matrix}$$

Define a vector \vec{qᵢ} that is perpendicular to \vec{vₗ} and pointed towards the center of the circle as \vec{qₗ}=(\vec{vₗ}×\vec{Vₗ₊₁})×\vec{vₗ}

Recognize that the center of the circle of rotation cᵢ is a distance Rᵢ from pᵢ along direction of \vec{qₗ}, and so

$$\vec{{c}_{ι}} = \vec{{p}_{ι}} + {R}_{i} \cdot \frac{\vec{{q}_{i}}}{\vert {q}_{i} \vert}$$

Determine the vector direction of the axis of rotation of the circle as

$$\vec{ω} = \frac{\vec{{v}_{ι}} \times \vec{{v}_{ι + 1}}}{\vert {v}_{i} \vert \vert {v}_{i + 1} \vert}$$

Given \vec{pl} as the position of sensor i, the center of the circle of rotation \vec{cₗ} between sensors i and i+1, the axis of rotation \vec{ω}, and the angle of rotation a, use the Euler-Rodrigues rotation method and operate that transformation on pᵢ to estimate the position of pᵢ₊₁ as:

$$\begin{matrix}a = cos ( \frac{α}{2} ) \\ b = {ω}_{x} sin ( \frac{α}{2} ) \\ c = {ω}_{y} sin ( \frac{α}{2} ) \\ d = {ω}_{Z} sin ( \frac{α}{2} ) \\ \vec{s} = ( b , c , d )\end{matrix}$$

and finally

\vec{pₗ₊₁}=\vec{pₗ}+(\vec{s}×\vec{pₗ})+2(\vec{s}×(\vec{s}×\vec{pₗ}))

Iterate through all measured values of \vec{vₗ} to estimate all values of pᵢ, and so estimate the shape of the shaft.

In some embodiments, a combination of the circle segment approximate algorithm and the linear approximation algorithm may be adopted depending on the arrangement or spacing of the sensor pairs. In some cases, the approximation algorithm may be selected based at least in part on the spacing between sensor pairs. For instance, when the spacing is greater than a predetermined threshold, the circle segment approximation may be selected and applied to the orientations to estimate the corresponding shape, and when the spacing is below the threshold, the linear approximation algorithm may be applied for the corresponding shape estimation.

### Improved Cubic Splines Algorithm for Estimating

In some embodiments, the shape of the flexible shaft may be estimated based on the series of orientations using an improved Cubic Splines Algorithm. The method may calculate shaft position from sequential orientation or derivative information based on the assumption that the shaft between sensors can be described as a cubic spline. This is a variation of a cubic approximation. This method can enforce the condition that the calculated position of each shaft segment meets at each sensor location, and also enforce the conditions that both the first and second derivatives of the shape of the shaft are identical where the shaft segments meet. Unlike conventional cubic spline fitting function that is to calculate the coefficients for a series of cubic spline functions that pass through a series of points in 3D, the improved algorithm herein may begin with the derivative of the curve at points along the curve as inputs rather than the position of the points themselves. By taking derivative of the curve instead of the position of the point, the algorithm herein may achieve an improved approximation accuracy (i.e., reducing fitting error).

In this method, as with most 3D parametric spline methods, the problem may be expressed separately in x, y, and z, and separately solved. The method may be uniquely applied to estimate the shaft shape by providing solutions that are coupled by a relationship between the first derivatives in the x, y, and z directions. With n+1 control points (p₀, p₁, . . . , pₙ), for each spline segment i there are three separate series of parametric equations:

xᵢ(t)=aₓᵢ+bₓᵢt+cₓᵢt²+dₓᵢt³

yᵢ(t)=a_{yi}+b_{yi}t+C_{yi}t²+d_{yi}t³

zᵢ(t)=a_{zi}+b_{zi}t+C_{zi}t²+d_{zi}t³

Where t is a parameter running from 0 to 1 and i=0, . . . , n−1 (with n+1 control points there are n spline segments between them). For each of x, y, and z there are n separate 3^{rd} order polynomial functions having 4 free parameters: a, b, c, d. Following the method of cubic splines as described in Bartels for interpolating cubic polynomial splines (but without reproducing each equation here) it is enforced that 1) the functions go through each control point at both the beginning and the end of the spline segment (giving two equations), 2) the first derivatives of functions from subsequent segments are equal at each control point (giving one equation), and 3) the second derivatives of functions from subsequent segments are equal at each control point (giving one equation). This results in four equations for the four unknown values, but with the equations from one segment mixed with the equations for the segments on either side, and lacking two equations associated with the end segments. The two lacking equations will be supplied by assuming particular boundary conditions, discussed further below, giving exactly as many equations as there are unknown quantities.

The above four equations may be rewritten using the set of x equations as an example (equations for y and z are identical), it can be found that:

aₓᵢ=xᵢ

bₓᵢ=Dₓᵢ

cₓᵢ=3(xᵢ₊₁−xᵢ)−2Dₓᵢ−Dₓ₍ᵢ₊₁₎

dₓᵢ=2(xᵢ−Xᵢ₊₁)+Dₓᵢ+Dₓ₍ᵢ₊₁₎ Eq. 1

With Dₓᵢ being the derivative of the function x(t), i.e.

$${D}_{xi} = \frac{\partial {x}_{i}}{\partial t} .$$

In order to supply two additional equations to have as many unique equations as there are unknowns it is required to make two assumptions regarding the boundary conditions at each end. There are many possible choices for assumptions regarding the boundary conditions that result in as many equations as there are unknowns, allowing a solution to be calculated. One common boundary condition used is the Hermite assumption that the second derivatives of the functions for the segments at each end are zero, e.g. for the functions for x (functions for y and z have identical form):

$$\frac{{\partial }^{2} {x}_{0} ( t = 0 )}{\partial {t}^{2}} = 0 and \frac{{\partial }^{2} {x}_{n - 1} ( t = 1 )}{\partial {t}^{2}} = 0$$

This is also a real-world physically relevant boundary condition to use because at either end of an endoscope within standard use the second derivative likely is in fact near 0.

Now that there are as many equations as there are unknowns the equations may be massaged into the form:

[21:141:141:14:1:1:41:12][Dₓ₀Dₓ₁Dₓ₂Dₓ₃:Dₓ₍ₙ₋₁₎Dₓₙ]=[3(x₁−x₀)3(x₂−x₁)3(x₃−x₂):3(xₙ₋₁−xₙ₋₃)3(xₙ−xₙ₋₂)3(xₙ−Xₙ₋₁)] Eq. 2

The method may use this equation with xᵢ as inputs, to solve for the Dₓᵢ values, and then to use the equations in Eq. 1 to find the values for aₓᵢ, bₓᵢ, cₓᵢ, dₓᵢ. This results in smooth parametrized curves that go through each point. In contrast to that typical use case, the method uses the Dₓᵢ values as inputs, solve for the xᵢ values, and with both Dₓᵢ and xᵢ known the method can recover the a, b, c, d values of Eq. 1.

One issue is how to begin with real world measured orientations and convert that into parametric space derivative values. As is shown in FIG. 3, for each sensor location the values (θᵢ, φᵢ) are measured by the device and provided. Projections of the orientation onto the x, y, and z axes are given by:

pₓᵢ=sin(θᵢ)cos(φᵢ),p_{yi}=sin(θᵢ)sin(φᵢ),p_{zi}=cos(θᵢ)

The space derivatives are given by the ratios of the projections as:

$$\begin{matrix}\frac{dy}{dx} = \frac{dz}{dx} , \frac{dz}{dx} = \frac{{p}_{z}}{{p}_{x}} \\ \frac{dy}{dx} and \frac{dz}{dx}\end{matrix}$$

are the inputs from the sensor used by the method as described here. For the spline functions in parametric space it requires

$$\frac{dx}{dt} , \frac{dy}{dt} , and \frac{dz}{dt} .$$

The relationship between

$$\frac{dy}{dx} , \frac{dz}{dx} , L and \frac{dx}{dt} , \frac{dy}{dt} , \frac{dz}{dt}$$

is established as follows:

For a space curve s parameterized from t=0 to t=1 with chord length L:

$$\begin{matrix}{\{ {dx}^{2} + {dy}^{2} + {dz}^{2} \}}^{\frac{1}{2}} = d = ds \\ {\{ {( \frac{dx}{dt} )}^{2} + {( \frac{dy}{dt} )}^{2} + {( \frac{dz}{dt} )}^{2} \}}^{\frac{1}{2}} = \frac{ds}{dt} = L \\ ( \frac{dx}{dt} ) {\{ 1 + \frac{{( \frac{dy}{dt} )}^{2}}{{( \frac{dx}{dt} )}^{2}} + \frac{{( \frac{dz}{dt} )}^{2}}{{( \frac{dx}{dt} )}^{2}} \}}^{\frac{1}{2}} = L \\ ( \frac{dx}{dt} ) {\{ 1 + {( \frac{dy}{dx} )}^{2} + {( \frac{dz}{dx} )}^{2} \}}^{\frac{1}{2}} = L\end{matrix}$$

And finally

$$\begin{matrix}( \frac{dx}{dt} ) = \frac{L}{{\{ 1 + {( \frac{dy}{dx} )}^{2} + {( \frac{dz}{dx} )}^{2} \}}^{\frac{1}{2}}} \\ ( \frac{dy}{dt} ) = ( \frac{dy}{dx} ) ( \frac{dx}{dt} ) = \frac{L ( \frac{dy}{dx} )}{{\{ 1 + {( \frac{dy}{dx} )}^{2} + {( \frac{dz}{dx} )}^{2} \}}^{\frac{1}{2}}} \\ ( \frac{dz}{dt} ) = ( \frac{dz}{dx} ) ( \frac{dx}{dt} ) = \frac{L ( \frac{dz}{dx} )}{{\{ 1 + {( \frac{dy}{dx} )}^{2} + {( \frac{dz}{dx} )}^{2} \}}^{1 / 2}}\end{matrix} \qquad Eq . 3$$

These three equations in Eq. 3 provide the relationship needed between

$$\frac{dy}{dx} , \frac{dz}{dx} , L and \frac{dx}{dt} , \frac{dy}{dt} , \frac{dz}{dt} .$$

With the orientation data of (θᵢ, φᵢ) from each sensor, the known spacing L; between sensors, and a starting position for the first sensor of (x₀, y₀, z₀), equations Eq. 1, Eq. 2, and Eq. 3 can be used to determine the position of all remaining sensors in three-dimensional space. The estimated accuracy of the methods is described later herein.

It should be noted that various other methods can be employed to estimate the shape of the shaft. For instance, a method to calculate shaft position from sequential orientation or derivative information may comprise utilizing splines to estimate position that are not cubic splines, or spline curves that are not required to pass strictly through the estimated control points, but rather any of many other possible spline estimation methods. As an example, B-splines (also called basis splines) are a generalization of polynomial splines, and may be used in ways similar to the ways described above for linear, circle section, or cubic splines to estimate the 3D position of the sensors and the shape of the shaft from derivatives at the sensor control points.

In some embodiments, the shape estimation algorithm may be based on physical constraints or property theory of the shaft. For instance, the method may calculate shaft position from sequential orientation or derivative information by utilizing specific knowledge of the response of the shaft to orientations in order to predict the shape from orientation. For example, a shaft may have a minimum bend radius, where bends down to some radius require very little force to achieve, but bends past a minimum bend radius require a great deal of force or perhaps can't practically be achieved. For example, for laser cut shafts, where horizontal cuts in the sidewall of tubes are made to allow bending, bends may be made with relatively low bending force until the laser cut section completely closes. Bending past that radius requires significantly more force than bends made down to that minimum bend radius. This specific physical constraints or property regarding the minimum bend radius may be used to improve the prediction of shape from orientation by disallowing predictions with bend radii beyond what is reasonably possible.

Other information that can be used to improve prediction of the shape of the shaft from orientation information is the functional form of the specific response of the shaft to orientation. The response of the shaft may be calculated and directly measured, and this information may be used to directly estimate the shaft shape from orientation information, or it may be used to set limits on or refine any of the previously described methods for predicting shaft shape from orientation. In some cases, the algorithm to further refine the estimated shape may be a machine learning algorithm trained model or by imposing conditions in an iterative optimization method.

In some embodiments, the method to calculate shaft position from sequential orientation or derivative information may comprise utilizing machine learning or neural networks to predict the shaft shape from derivative information. The machine learning algorithm can be any type of machine learning network such as a neural network. Examples of neural networks include a deep neural network, a convolutional neural network (CNN), and a recurrent neural network (RNN). The machine learning algorithm may comprise one or more of the following: a support vector machine (SVM), a naïve Bayes classification, a linear regression model, a quantile regression model, a logistic regression model, a random forest, a neural network, CNN, RNN, a gradient-boosted classifier or repressor, or another supervised or unsupervised machine learning algorithm (e.g., generative adversarial network (GAN), Cycle-GAN, etc).

In some cases, the model may be trained using supervised learning. The training data sets may comprise pairs of input data (e.g., sequential orientation or derivative information) and ground truth data (e.g., shape data). In some cases, a training dataset is created using shaft and sensor data sets that are substantially representative of the shafts and sensors to be used endoscopically. The training dataset may be created by moving and contorting representative shafts through a broad range of positions, acquiring sensor orientation information (as input data) and also independently knowing or measuring the corresponding shaft position (ground truth). In some cases, different shafts with different mechanical properties (e.g., stiffness, dimension, materials, etc) may be utilized to obtain the training data. For instance, various shaft shapes and the paired positions are obtained from various different shafts. The positions chosen may be random, or the shaft may be moved systematically. The positions chosen may be randomized, but drawn from the set of positions generally expected to be encountered by an endoscope. The position of the shaft may be robotically controlled or may be achieved by other means. Independently determining the position along the shaft may be accomplished by knowing the position of the end effector of robotic actuators clamped to the shaft and used to move the shaft through the broad range of positions. In some cases, independently determining the position along the shaft (i.e., ground truth) may be accomplished by instrumenting the shaft, e.g., with EM sensors, or optical fiber based position sensors, or a camera or cameras able to track position along the length of the shaft in 3D using computer vision algorithms, or other means. In some cases, after a model is trained, the parameters of the model may be continuously updated based on newly acquired sensor data. In some cases, after a model is trained, the parameters of the model may be continuously updated based on feedback data. For example, a difference between a model predicted shape/position along the shaft and a reference shape/position may be used as feedback data to automatically update the model. The reference shape/position may be acquired utilizing the robotic command, a simulation analysis or other sensors (e.g., external sensors or EM sensor) as described above.

In some cases, the system may monitor data drift or performance of a model in different phases (e.g., development, deployment, prediction, validation, etc.). The system may be configured to perform data/model integrity checks and detect data drift and accuracy degradation. The process may begin with detecting data drift in training data and prediction data. During training and prediction, the model monitor system may monitor difference in distributions of training data, test, validation and prediction data, change in distributions of training data, test, validation and prediction data over time, covariates that are causing changes in the prediction output, and various others. Data monitored by the model monitor system may include data involved in model training and during production. The data at model training may comprise, for example, training, test and validation data, predictions, or statistics that characterize the above datasets (e.g., mean, variance and higher order moments of the data sets). Data involved in production time may comprise time, input data, predictions made, and confidence bounds of predictions made. In some embodiments, the ground truth data (e.g., positions or shape along a shaft) may also be monitored. The ground truth data may be monitored to evaluate the accuracy of a model and/or trigger retraining of the model. The system may monitor changes in data such as changes in ground truth data, or when new training data or prediction data becomes available.

In some cases, the performance (e.g., prediction accuracy) of the model may be monitored and upon detection of the performance is below a threshold, the model may be automatically retrained or updated. The performance monitoring may be based on the feedback data as described above. In some cases, the performance monitoring may be based on a difference/error between the model predicted shape/positions along the shaft and a reference shape. The reference shape may be obtained utilizing other sensor data. For example, imaging modalities such as computed tomography (CT), ultrasound, tomosynthesis or fluoroscopy employed during a surgery operation may be used to acquire image of the target site and a reference shape of the shaft may be obtained from a segmentation of the shaft from the image data. A predicted shape from the model may be compared against the shape from the image data to determine a performance of the model. In some cases, the feedback loop may be implemented by adding a feedback ingestion loop to the model network. In some cases, to speed up the retraining process, only the last layer of the convolutional neural network (CNN) model is updated/retrained utilizing the reference shape data (upon determining the error is above a threshold) while keeping the weights of all other layers intact. In the case of CNN, the process may comprise extracting the features using the model but slightly adjusting the classifier to account for the error. Alternatively, the feedback loop may be implemented by employing a simple linear logistic regression model to keep learning and prediction scalable based on the reference shape data upon detection of model drift then combine the results of the predictions from the CNN and the simple linear logistic regression model.

### Performance Evaluation of the Shape Sensing Methods

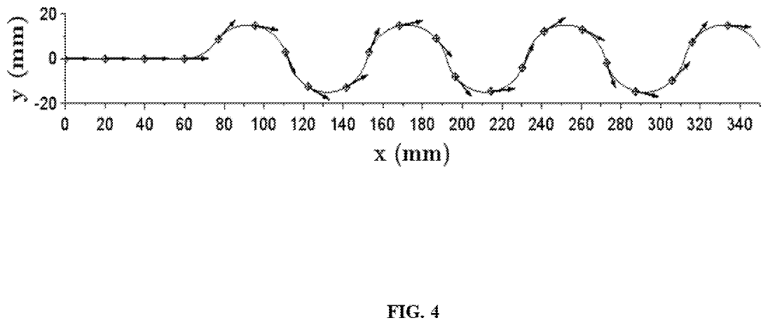

FIG. 4 shows a specific shape used for estimating system performance via simulation. In the example, the total sensed length is 420 mm simulated the performance of the system using the mathematical method of circle segments to estimate the position of the shaft from the sequence of orientations along the shaft, using the specific shape shown in FIG. 4. The evaluation includes assessing how the estimation of position is impacted by 1) the spacing between sensor pairs, 2) the minimum bend radius Rbend of the shaft, 3) error in measured angles, e.g. from sensor noise, and 4) error in the spacing L between sensors, e.g. from construction. The simulation is performed in three-dimension (3D). The illustrated example shows an example of the shaft bent or articulated in the x-y plane, the z-dimension is considered in the case where angular errors are simulated. The errors shown below are the max error in 3D at any point along the shaft. The maximum errors are almost always at distal end of the shaft.

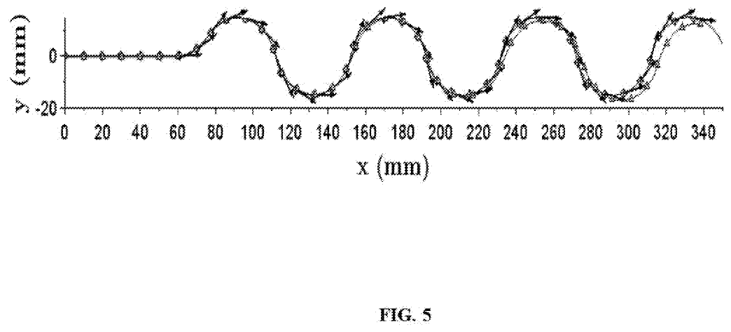

FIG. 5 shows an example of the output of the simulation using the circle segment approximation. In the example of the output of a simulation, the minimum bend radius is 20 mm, the spacing between sensors is 10 mm, there is 0.5 degrees standard deviation error in measuring the angles, and there is 0.25 mm standard deviation in the spacing between sensors. The diamonds indicators represent the “true” position of the sensors and the triangles indicators represent the location estimated by the circle section method. Max 3D error for this simulation is 5.2 mm over the 420 mm sensed length.

Based on the evaluation, it is found that the error in estimating position scales with the ratio of (sensor spacing)/(avg bend radius). If the sensor spacing L<0.5*R_{bend} then the maximum 3D error at any point is less than about 5 mm (~<5) mm over a 420 mm length for the shaft shape in FIG. 5.

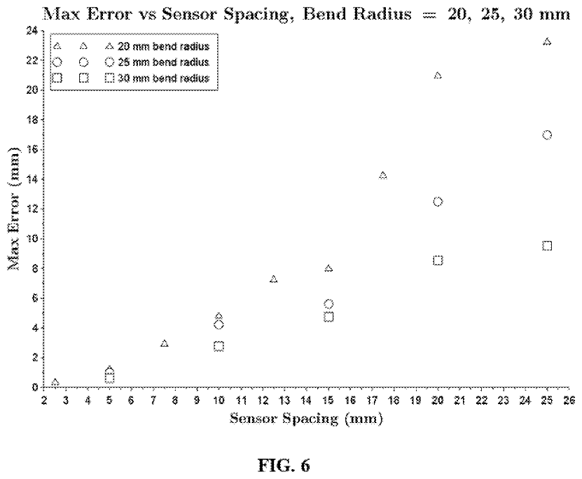

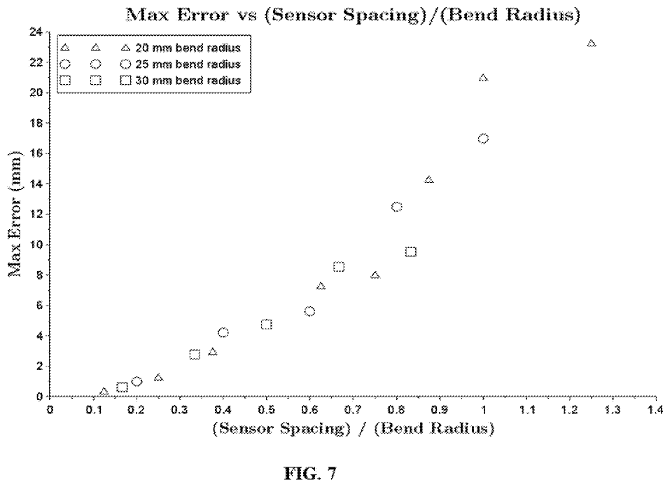

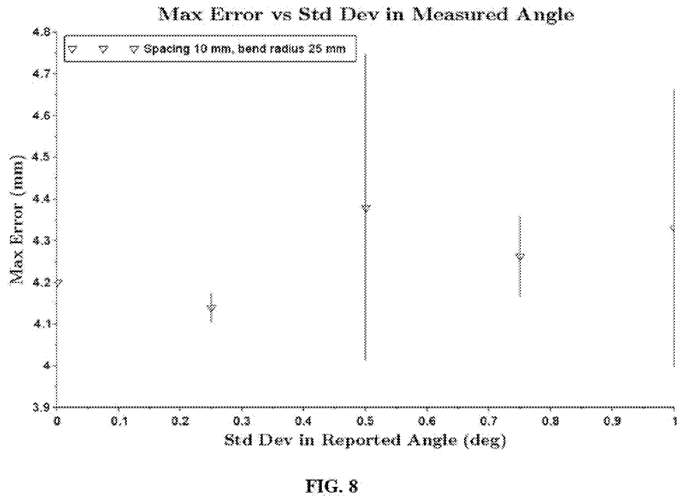

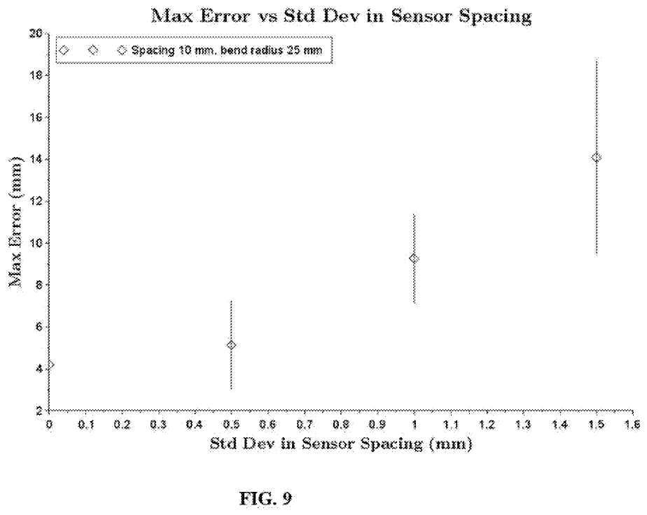

FIG. 6 shows error in location as a function of sensor spacing for shafts with three different radii of curvatures and FIG. 7 shows the error in location as a function of sensor spacing divide by the avg radius of curvature. This result shows that the sensor spacing should be about half or 0.5x the expected radius of curvature for reasonable or sufficient accuracy. Additionally, FIG. 8 shows error in location as a function of standard deviation in the measurement of the orientation angle for each sensor indicating that the estimation error has only a weak dependence with error in the measured angle. FIG. 9 shows error in location as a function of standard deviation in the spacing between sensors along the shaft indicating that the estimation error has a strong dependence with error in the spacing between sensors.

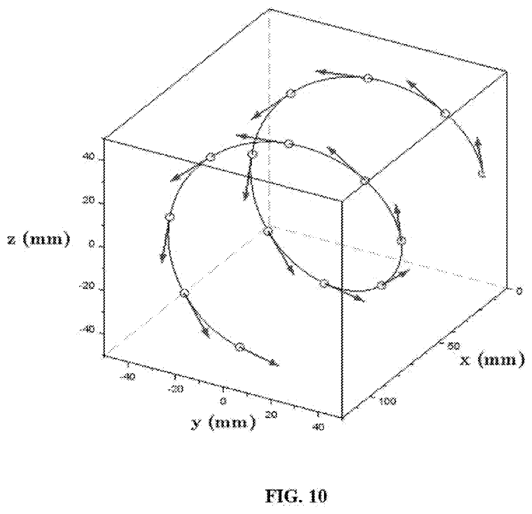

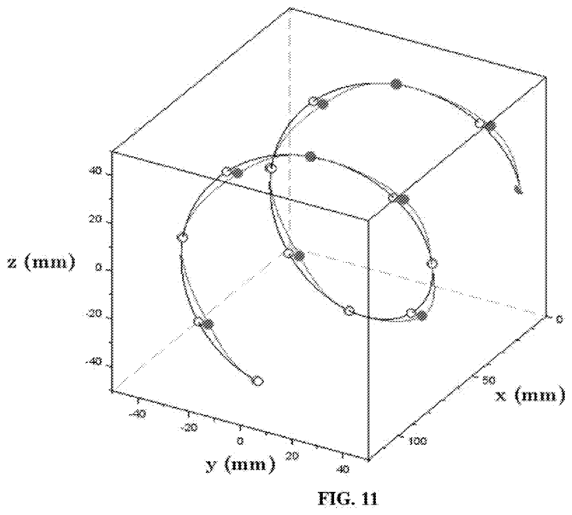

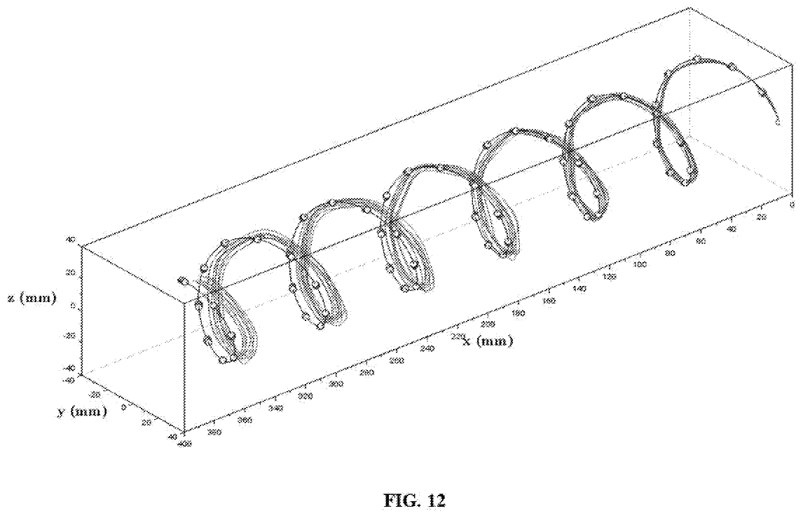

FIGS. 10-12 show performance of the cubic splines-based estimation method herein. The simulated the performance of the system using the mathematical method of circle segments to estimate the position of the shaft from the sequence of orientations along the shaft, using the specific shape are shown in the figures. FIG. 10 shows an example shape with control points and measured derivative values at control points. In FIG. 11, the circles indicators represent control points as in FIG. 10, and dots indicators represent the calculated location of the control points using the method of cubic splines for the position estimates. FIG. 12 shows a representative series of monte Carlo simulations for a full 1.2 m length shaft, with a radius of curvature close to 20 mm, with assumed levels of error in measuring the angle at each sensor location and in placement of the sensors along the shaft. The simulation results from 10 monte Carlo runs overlayed with true control point locations for the conditions: total shaft length=1200 mm, sensor spacing L=20 mm, std dev of angle measurement δθ=1\hat{ }^{o}, std dev of error in placement of sensors.

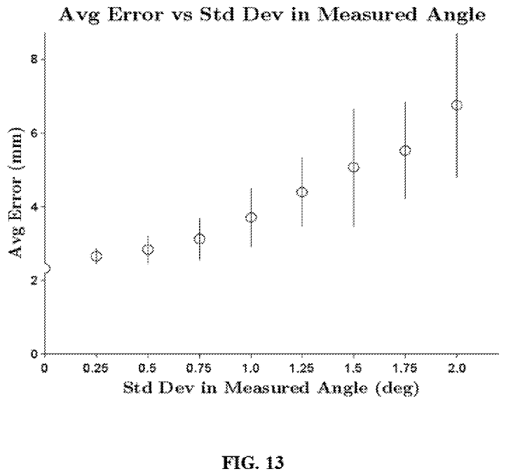

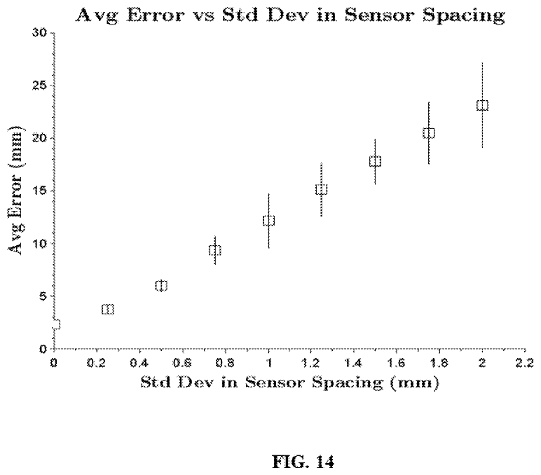

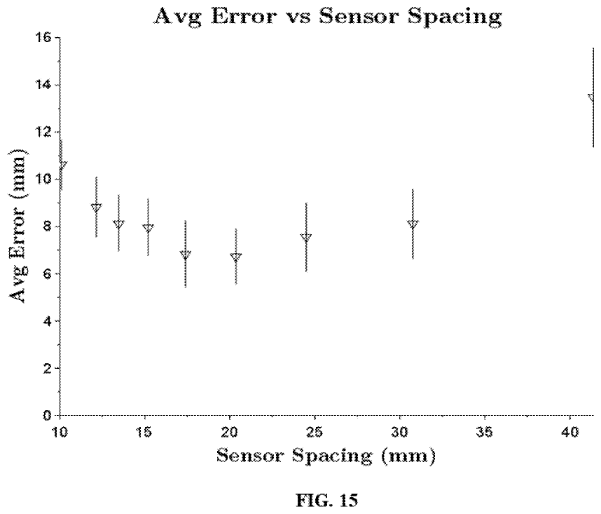

FIGS. 13-15 show the simulated system performance as a function of the standard deviation in the measurement angle (FIG. 13), the standard deviation in the spacing between sensors along the shaft (FIG. 14), and the sensor spacing (FIG. 15). FIG. 13 shows the avg error across 20 monte Carlo runs for the location of the last sensor on a 1200 mm shaft as a function of std dev of error in measurement angle given the conditions: sensor spacing L=20 mm, std dev of error in placement of sensors along the length 8L-0 mm, period along x=60 mm, radius in yz=30 mm.

FIG. 14 shows the avg error across 20 monte Carlo runs for the location of the last sensor on a 1200 mm shaft as a function of std dev of error in placement of sensors along the length given the conditions: sensor spacing L=20 mm, std dev of angle measurement δθ=0\hat{ }^{o}, period along x=60 mm, radius in yz=30 mm. FIG. 15 shows the avg error across 20 monte Carlo runs for the location of the last sensor on a 1200 mm shaft as a function of sensor spacing given the conditions: std dev of angle measurement δθ=0\hat{ }^{o}, std dev of error in placement of sensors along the length is the x-axis, period along x=60 mm, radius in yz=30 mm.

Improving the Density of Sensor Measurements without Increasing the Number of Sensors

The above evaluation methods show that estimation error improves (limited by the minimum bending radius) when the measurements of the orientation are spaced more closely to each other. Conventional methods would require additional sensors to be incorporated in the device to reduce the spacing thereby reducing the shape estimation error. The present disclosure provides methods to improve the estimation error (decreasing the estimation error) in calculating shaft position from sequential orientation or derivative information without increasing the number of sensors. In some embodiments, the method may achieve an effective density of measurement position that is greater than the density of sensor spacing by placing the sensors on a structure which is actuated to slide forwards and backwards relative to an axial axis direction of the endoscope and is within the endoscope, and to make measurements as the sensor structure is moved. By coordinating a movement of the sensors relative to the shaft and the sensor measurements, the effective density of measurement positions is increased. For example, if the sensors are spaced 20 mm apart, and the sensor structure is moved+/−10 mm (20 mm total amplitude of travel) with measurements made with every 1 mm of movement, then the orientation of the shaft has effectively been measured with a spacing between measurements of 1 mm using sensors that are spaced by 20 mm. In some cases, the density of measurement positions or measurement density is adjustable (increased/decreased) by controlling a movement of the movable structure holding the sensors and/or the sensor measurement frequency.

In some embodiments, the slidable structure that the sensors are mounted to may be actuated to move along the axial axis of the shaft in incremental steps. In some cases, the slidable structure may be actuated to move in accordance with the sensor measurements such that the sensor measurements are obtained at each step. Alternatively, the slidable structure may be actuated to move in continuous motion with sensor measurements acquired at pre-determined frequency such that the motion results in only a small amount of positional averaging of the orientation. In some cases, the accelerometer sensor reading may be corrected for the effect of acceleration of the slidable structure on the sensor measurements (i.e., accelerometer reading) that is used to determine orientation. In some cases, when the sensor measurements are made while the sensor/slidable structure is being accelerated, the acceleration vector of the slidable structure motion may be corrected from the accelerometer sensor reading. In some cases, the slidable structure motion may be controlled to move at constant velocity during sensor measurement to not introduce acceleration. For example, the slidable structure motion may be a “triangle wave” where at the end of the amplitude of travel the sensor structure is quickly decelerated/accelerated to go from positive velocity (+velocity) to negative (-velocity), and is held at the new velocity until reaching the opposite amplitude of travel. In some cases, even at constant velocity the slidable structure motion may experience acceleration due to non-linearity of the shaft (e.g., at bends of the shaft there will be associated accelerations). The methods herein may solve the above issue (i.e., acceleration due to non-linear shaft) by pre-calculating impact of accelerations (change of velocity in magnitude and/or direction) at different points along the shaft with various non-linear shapes on the expected sensor readings. In some cases, the system or method herein may determine the shape by self-consistently or iteratively solving for the shape, given the shape calculating the impact of accelerations at different points along the shaft on the expected sensor readings, and correcting for the impact from the sensor measurements.

In some embodiments, the method may improve the estimation error by controlling the spacing between sensors with a low standard deviation of placement error. The methods and systems herein may employ unique means for accurately positioning the sensors along the structure holding the sensors (e.g., slidable structure). In some cases, the slidable structure and the placement of sensors on the slidable structure are actuated to have translational motion relative to the axial axis of the shaft while the sensors are affixed to the slidable structure. In some cases, the slidable structure may comprise a cable that can be actuated to translate within the shaft by actuators located at the proximal end of the endoscope. In some cases, the slidable structure may comprise a plurality of mounting structures for coupling the sensors to the cable.

In some embodiments, the sensors are placed at known spacings by being soldered onto long flexible cables that have mounting points fabricated lithographically to be in accurate locations along the length of the cable. In some embodiments, the sensors are placed at known spacings by mechanical structures fabricated into the shaft. In some cases, all of the sensors for estimating the shape are affixed to the slidable structure. In some cases, at least a subset of the sensors are affixed to the slidable structure. For instance, a subset of the sensors at particular segment along the length of the shaft may be mounted to the slidable structure to locally increase the effective density of measurement positions.

In some embodiments, the sensors are placed at known spacings by mechanical structures molded into a flexible injection molded form. In some embodiments, the sensor spacing is not constant along the length, with closer spacing used for regions where greater accuracy and precision are desired.

In some embodiments, the effective number of points of measurement of orientation along the shaft is increased by having a structure (i.e., slidable structure) to which the sensors are mounted able to move forwards and backwards along the shaft being instrumented (distally and proximally), with measurements of orientation made with the sensor structure held at multiple positions along the shaft. For example, if the sensors are spaced 20 mm apart from one another, and if the sensors are mounted to a structure that can be moved back and forth+/−10 mm (20 mm total distance) within the shaft to be measured, and if measurements are made at every 1 mm change in position, then the effective sensor spacing for estimation of the shaft shape is 1 mm whereas sensors are physically separated by 20 mm. In some cases, the forwards and backwards movement is continuous and positions along the shaft are measure by timing when the measurements are made with respect to the motion of the structure. In some cases, the forwards and backwards movement is sinusoidal in time. In some cases, the forwards and backwards movement is a triangle wave in time.

In some embodiments, the sensors may be calibrated at initialization of the instrumented shaft. In some cases, the instrumented shaft is provided in packaging (e.g., in the as-shipped state) that holds the shaft in a controlled and known shape. At the time of use, the shaft is first initialized while still within the packaging in order to calibrate the orientation of all sensors with respect to the known shape of the shaft.

In some embodiments, the instrumented shaft is mechanically designed to have a known shape with a given orientation at positions along the shaft, allowing an improved estimation of position from orientation.

The estimated shape of at least a portion of the shaft can provide various advantages as described elsewhere herein. The estimated shape of shaft can be used for navigating an endoscope along an airway or passageway. For example, when the endoscope is within the lung, and from preoperative CT and segmentation the shape of the airways within the lung are known, and if the shape of a significant length of the endoscope within the body is known by the methods disclosed here, then by matching the shape of the endoscope to the possible airway shapes within the lung it can determine where within the lung the endoscope is located and to track the motion of the endoscope by continually matching the measured shape of the endoscope to the shape of the passageways. This allows the endoscope to be navigated through the physiology. In another example, when the shape of the control region of the endoscope is known, the shape information may be used as feedback for robotic control. For instance, the estimated shape information may be used in feedback control when it is desired for the distal end of the endoscope to be held fixed with respect to a lesion during a procedure, or when it is desired to automatically and robotically navigate through physiology.

In some embodiments, a calibration method or calibration algorithm is provided to determine to high accuracy the position and orientation of each sensor along the length of a shaft. In some cases, calibrating the series of sensor pairs based on a first known shape of the elongated member and a first corresponding series of orientations, and a second known shape of the elongated member and a second corresponding series of orientations. In some embodiments, the calibration method may comprise first, placing the shaft into a structure that holds the shaft accurately in a known shape and obtaining the output reading of the sensors. Second, placing the shaft into a structure that holds the shaft accurately in a second known shape, and obtaining the output readings of the sensors when the shaft is held in the second shape. The first shape and the second shape of the shaft may be different along the length (i.e., bending, curvature, orientation, position and the like are different). Next, the calibration method may comprise combing the shape data from the two known shapes and the sensor readings/measurements acquired when the shaft is held at the two shapes to calculate a) the exact position of the sensors along the shaft, and b) the orientation of the sensors with respect to the shaft.

In some cases of the calibration method, a first shape may be a straight shaft (i.e., straight configuration), and measurement of the sensor readings when held in a straight orientation allows the orientation of the sensor with respect to the shaft to be calculated, and a second shape is a cylinder or arc shape with a known diameter/radius or curvature (i.e., non-straight configuration). With the sensor orientations with respect to the shaft known from the first shape, and second shape, from the orientation data when held in the second shape, the position of the sensors along the shaft can be accurately calculated.

In some embodiments, calibration data about the position and orientation of each sensor along the shaft is stored in a memory device. In some cases, the memory device may be incorporated into the shaft, and at the time of use is read by a device that takes the data from the shaft and calculates the shape. For example, the memory device may store the calibration data and upon initialization, the memory device may establish wired or wireless communication with a controller of the endoscope for the controller to receive the calibration data. The controller may be located at a handle portion of the endoscope. Alternatively, the controller may be located at the instrument driving mechanism (IDM) or a robotic support system and may be connected to the memory device via the electronic interface as described later herein.

The present disclosure provides methods and systems for obtaining a shape of at least a portion of a flexible shaft of an endoscope. In some embodiments, the method may calculate the shaft position by using a linear approximation based on the shaft derivative information.

In some embodiments, the method may calculate the shaft position is by using a circle section approximation based on the shaft derivative information.

In some embodiments, the method may calculate the shaft position is by using a cubic spline approximation based on the shaft derivative information.

In some embodiments, the accelerometer and/or magnetometer data are tracked in real-time to detect if the endoscope is likely in motion and/or if it is likely accelerating. Such motion information may be taken into account when calculating the shape. For instance, based on the motion data, if the endoscope is moving or accelerating, shape calculation may not be performed until the endoscope is at rest or is at constant velocity. In some cases, the acceleration or motion data may be used for correcting the final estimated shape. For example, based on the acceleration or motion data, corrections due to the acceleration or motion may be made.

In some embodiments, the method to use the shaft derivative information to calculate the shaft position uses specific information regarding the mechanical response of the shaft to being held at different orientations.

In some embodiments, the method to use the shaft derivative information to calculate the shaft position is to use a machine learning algorithm or neural network, with the steps of i) acquire a training dataset for position along the shaft as a function of orientation along the shaft, 2) train the machine learning or neural network algorithm to be able to take in orientation information and predict shape information, and 3) and use that machine learning or neural network to predict shape from measured orientation information.

### Flexible Endoscope System and Device

In some embodiments, the methods and systems herein may be utilized for improving reliability and accuracy of controlling an operation of a flexible endoscope.

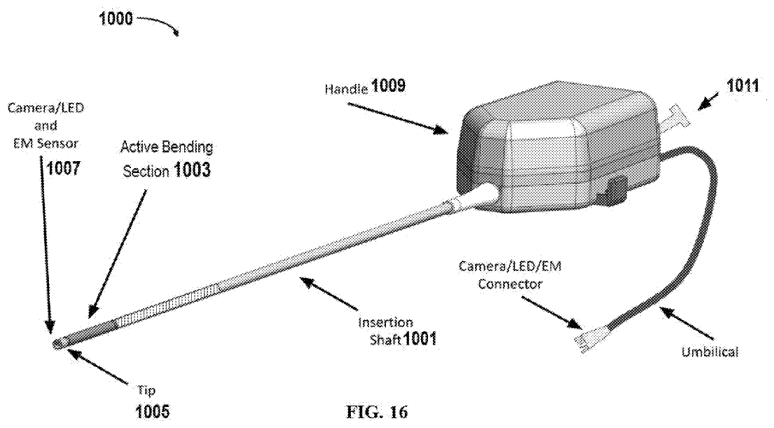

FIG. 16 illustrates an example of a flexible endoscope 1000, in accordance with some embodiments of the present disclosure. As shown in FIG. 10, the flexible endoscope 1000 may comprise a handle/proximal portion 1009 and a flexible elongate member to be inserted inside of a subject. The flexible elongate member may comprise a shaft described above. In some embodiments, the flexible elongate member may comprise a proximal shaft (e.g., insertion shaft 1001), steerable tip (e.g., tip 1005), and a steerable section (active bending section 1003). The shape sensing method and mechanism as described above may be applied to the insertion shaft, the bending section of a combination of both. The active bending section, and the proximal shaft section can be the same as those described elsewhere herein. The endoscope 1000 may also be referred to as steerable catheter assembly as described elsewhere herein. In some cases, the endoscope 1000 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiment, the endoscope may contain varying levels of stiffness along the shaft, as to improve functional operation.

The endoscope or steerable catheter assembly 1000 may comprise a handle portion 1009 that may include one or more components configured to process image data, provide power, or establish communication with other external devices. For instance, the handle portion may include a circuitry and communication elements that enables electrical communication between the steerable catheter assembly 1000 and an instrument driving mechanism (not shown), and any other external system or devices. In another example, the handle portion 1009 may comprise circuitry elements such as power sources for powering the electronics (e.g., camera, electromagnetic sensor and LED lights) of the endoscope. In some embodiments, the circuitry elements may comprise a memory device storing the calibration data of the sensors as described above.

The one or more components located at the handle may be optimized such that expensive and complicated components may be allocated to the robotic support system, a hand-held controller or an instrument driving mechanism thereby reducing the cost and simplifying the design the disposable endoscope. The handle portion or proximal portion may provide an electrical and mechanical interface to allow for electrical communication and mechanical communication with the instrument driving mechanism. The instrument driving mechanism may comprise a set of motors that are actuated to rotationally drive a set of pull wires of the catheter. The handle portion of the catheter assembly may be mounted onto the instrument drive mechanism (IDM) so that its pulley/capstans assemblies are driven by the set of motors. The number of pulleys may vary based on the pull wire configurations. In some cases, one, two, three, four, or more pull wires may be utilized for articulating the flexible endoscope or catheter. In some embodiments, at least a cable may be used for moving the sensors along the shaft for increasing the effective density of the sensors for shape estimation as describe above. The cable may be actuated by a pulley located at the IDM.

The handle portion may be designed allowing the robotic bronchoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

The electrical interface (e.g., printed circuit board) may allow image/video data and/or sensor data to be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals. As describe above, the circuitry elements may comprise a memory device storing the calibration data of the sensors and the electrical interface may allow the calibration data to be transmitted to the circuitry located at the IDM for calibrating the sensor readings.

In some cases, the handle portion 1009 may comprise one or more mechanical control modules such as lure 1011 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 1000. The mechanical interface may allow the steerable catheter assembly 1000 to be releasably coupled to the instrument driving mechanism. For instance, the handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor) 1007 is located at the tip of the catheter or endoscope shaft 1005. For example, line of sight of the camera may be controlled by controlling the articulation of the active bending section 1003. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with aid of an optimal component.

The distal tip 1005 may be a rigid component that allow for positioning sensors such as electromagnetic (EM) sensors, imaging devices (e.g., camera) and other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5D or 6D space. This may provide a visual guide to an operator when driving the bronchoscope towards the target site.

The endoscope may have a unique design in the elongate member. In some cases, the active bending section 1003, and the proximal shaft of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

As described above, the active bending section 1003 may be designed to allow for bending in two or more degrees of freedom (e.g., articulation). A greater bending degree such as 180 and 270 degrees (or other articulation parameters for clinical indications) can be achieved by the unique structure of the active bending section. In some cases, a variable minimum bend radius along the axial axis of the elongate member may be provided such that an active bending section may comprise two or more different minimum bend radii.

The articulation of the endoscope may be controlled by applying force to the distal end of the endoscope via one or multiple pull wires. The one or more pull wires may be attached to the distal end of the endoscope. In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley may interact with an output shaft from the robotic system.

In some embodiments, the proximal end or portion of one or more pull wires may be operatively coupled to various mechanisms (e.g., gears, pulleys, capstans, etc.) in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons or biocompatible material. Pull wires may be a wire, cable or a thread. In some embodiments, different pull wires may be made of different materials for varying the load bearing capabilities of the pull wires. In some embodiments, different sections of the pull wires may be made of different material to vary the stiffness and/or load bearing along the pull. In some embodiments, pull wires may be utilized for the transfer of electrical signals.

The proximal design may improve the reliability of the device without introducing extra cost allowing for a low-cost single-use endoscope. In another aspect of the invention, a single-use robotic endoscope is provided. The robotic endoscope may be a bronchoscope and can be the same as the steerable catheter assembly as described elsewhere herein. Traditional endoscopes can be complex in design and are usually designed to be re-used after procedures, which require thorough cleaning, dis-infection, or sterilization after each procedure. The existing endoscopes are often designed with complex structures to ensure the endoscopes can endure the cleaning, dis-infection, and sterilization processes. The provided robotic bronchoscope can be a single-use endoscope that may beneficially reduce cross-contamination between patients and infections. In some cases, the robotic bronchoscope may be delivered to the medical practitioner in a pre-sterilized package and are intended to be disposed of after a single-use.

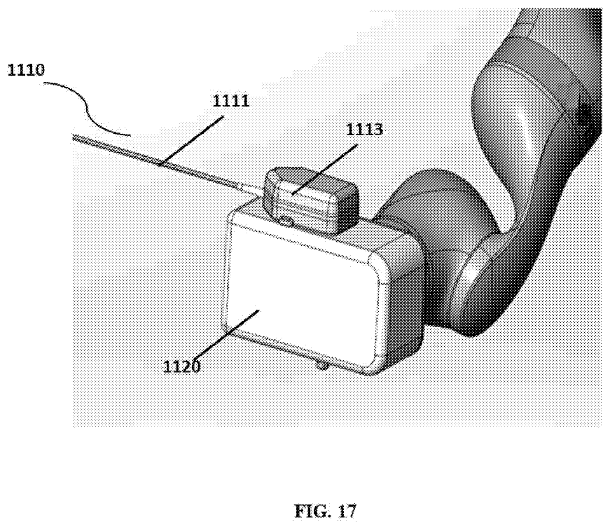

As shown in FIG. 17, a robotic bronchoscope 1110 may comprise a handle portion 1113 and a flexible elongate member 1111. In some embodiments, the flexible elongate member 1111 may comprise a shaft, steerable tip, and a steerable/active bending section. The robotic bronchoscope 1110 can be the same as the steerable catheter assembly as described in FIG. 16. The robotic bronchoscope may be a single-use robotic endoscope. In some cases, only the catheter may be disposable. In some cases, at least a portion of the catheter may be disposable. In some cases, the entire robotic bronchoscope may be released from the instrument driving mechanism and can be disposed of. In some cases, the bronchoscope may contain varying levels of stiffness along its shaft, as to improve functional operation. In some cases, a minimum bend radius along the shaft may vary.

The robotic bronchoscope can be releasably coupled to an instrument driving mechanism 1120. The instrument driving mechanism 1120 may be mounted to the arm of the robotic support system or to any actuated support system as described elsewhere herein. The instrument driving mechanism may provide mechanical and electrical interface to the robotic bronchoscope 1110. The mechanical interface may allow the robotic bronchoscope 1110 to be releasably coupled to the instrument driving mechanism. For instance, the handle portion of the robotic bronchoscope can be attached to the instrument driving mechanism via quick install/release means, such as magnets and spring-loaded levels. In some cases, the robotic bronchoscope may be coupled or released from the instrument driving mechanism manually without using a tool.

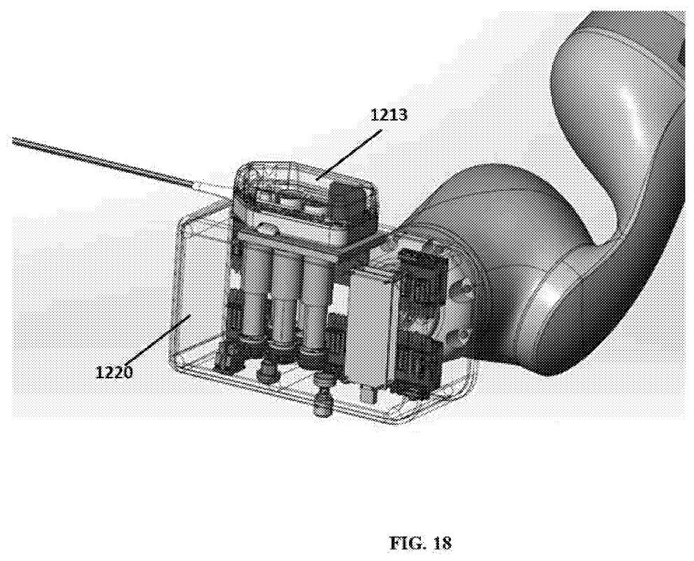

FIG. 18 shows an example of an instrument driving mechanism (IDM) 1220 providing mechanical interface to the handle portion 1213 of the robotic bronchoscope. As shown in the example, the instrument driving mechanism 1220 may comprise a set of motors that are actuated to rotationally drive a set of pull wires of the flexible endoscope or catheter. In some embodiments, at least one of the actuators/motors is used to acuate a slidable structure for translating a series of sensors along the shaft as described above. The handle portion 1213 of the catheter assembly may be mounted onto the instrument drive mechanism so that its pulley assemblies or capstans are driven by the set of motors. The number of pulleys may vary based on the pull wire configurations. In some cases, one, two, three, four, or more pull wires may be utilized for articulating the flexible endoscope or catheter.

The handle portion may be designed allowing the robotic bronchoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive, adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

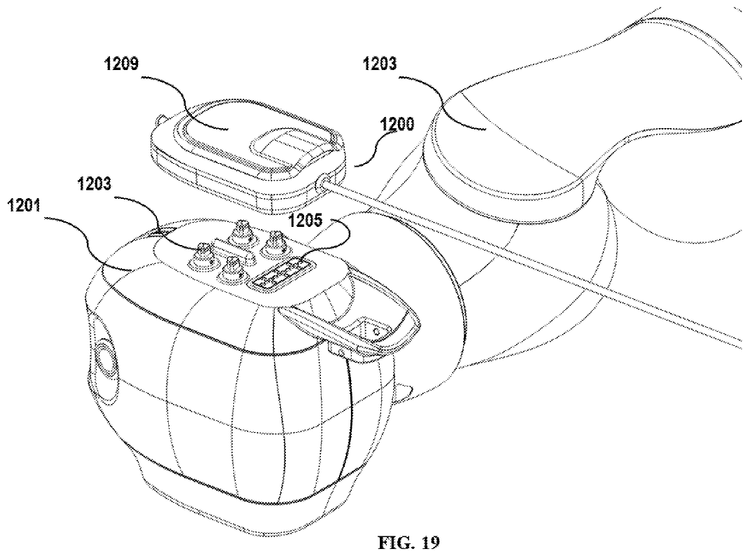

FIG. 19 shows another example of a disposable endoscope 1200 removably coupled to an IDM 1201. The one or more components located at the handle 1209 may be optimized such that expensive and complicated components may be allocated to the robotic support system 1203, a hand-held controller or an instrument driving mechanism 1201 thereby reducing the cost and simplifying the design the disposable endoscope. The handle portion or proximal portion 1209 may provide an electrical interface and mechanical interface to allow for electrical communication and mechanical communication with the instrument driving mechanism 1201. The instrument driving mechanism 1201 may comprise a set of motors that are actuated to rotationally drive a set of pull wires of the catheter. The handle portion 1209 of the catheter assembly may be mounted onto the instrument drive mechanism 1201 so that its pulley/capstans assemblies are driven by the set of motors. For example, the handle pulley may interact with an output shaft 1203 from the IDM supported by the robotic system The number of pulleys may vary based on the pull wire configurations. In some cases, one, two, three, four, or more pull wires may be utilized for articulating the flexible endoscope or catheter.

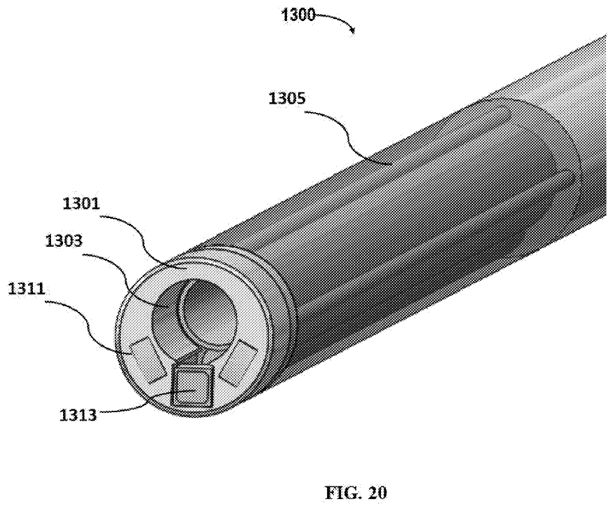

FIG. 20 shows an example of a distal tip 1300 of an endoscope. In some cases, the distal portion or tip of the catheter 1300 may be substantially flexible such that it can be steered into one or more directions (e.g., pitch, yaw). The catheter may comprise a tip portion, bending section, and insertion shaft. In some embodiments, the catheter may have variable bending stiffness along the longitudinal axis direction. For instance, the catheter may comprise multiple sections having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some embodiments, the catheter may have variable minimum bend radius along the longitudinal axis direction. The selection of different minimum bend radius at different location long the catheter may beneficially provide anti-prolapse capability while still allow the catheter to reach hard-to-reach regions. In some cases, a proximal end of the catheter needs not be bent to a high degree thus the proximal portion of the catheter may be reinforced with additional mechanical structure (e.g., additional layers of materials) to achieve a greater bending stiffness. Such design may provide support and stability to the catheter. In some cases, the variable bending stiffness may be achieved by using different materials during extrusion of the catheter. This may advantageously allow for different stiffness levels along the shaft of the catheter in an extrusion manufacturing process without additional fastening or assembling of different materials.

The distal portion of the catheter may be steered by one or more pull wires 1305. The distal portion of the catheter may be made of any suitable material such as co-polymers, polymers, metals or alloys such that it can be bent by the pull wires. In some embodiments, the proximal end or terminal end of one or more pull wires 1305 may be coupled to a driving mechanism (e.g., gears, pulleys, capstan etc.) via the anchoring mechanism as described above.

The pull wire 1305 may be a metallic wire, cable or thread, or it may be a polymeric wire, cable or thread. The pull wire 1305 can also be made of natural or organic materials or fibers. The pull wire 1305 can be any type of suitable wire, cable or thread capable of supporting various kinds of loads without deformation, significant deformation, or breakage. The distal end or portion of one or more pull wires 1305 may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The catheter may have a dimension so that one or more electronic components can be integrated to the catheter. For example, the outer diameter of the distal tip may be around 4 to 4.4 millimeters (mm), and the diameter of the working channel may be around 2 mm such that one or more electronic components can be embedded into the wall of the catheter. However, it should be noted that based on different applications, the outer diameter can be in any range smaller than 4 mm or greater than 4.4 mm, and the diameter of the working channel can be in any range according to the tool dimensional or specific application.

The one or more electronic components may comprise an imaging device, illumination device or sensors. In some embodiments, the imaging device may be a video camera 1313. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 1311 positioned at the distal tip. The light source may be a light-emitting diode (LED), an organic LED (OLED), a quantum dot, or any other suitable light source. In some cases, the light source may be miniaturized LED for a compact design or Dual Tone Flash LED Lighting.

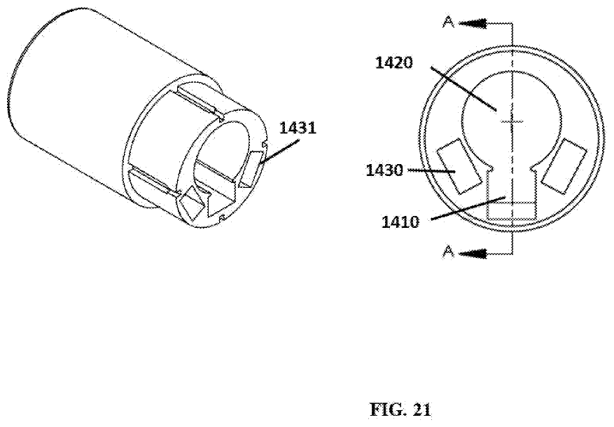

The imaging device and the illumination device may be integrated to the catheter. For example, the distal portion of the catheter may comprise suitable structures matching at least a dimension of the imaging device and the illumination device. The imaging device and the illumination device may be embedded into the catheter. FIG. 21 shows an example distal portion of the catheter with integrated imaging device and the illumination device. A camera may be located at the distal portion. The distal tip may have a structure to receive the camera, illumination device and/or the location sensor. For example, the camera may be embedded into a cavity 1410 at the distal tip of the catheter. The cavity 1410 may be integrally formed with the distal portion of the cavity and may have a dimension matching a length/width of the camera such that the camera may not move relative to the catheter. The camera may be adjacent to the working channel 1420 of the catheter to provide near field view of the tissue or the organs. In some cases, the attitude or orientation of the imaging device may be controlled by controlling a rotational movement (e.g., roll) of the catheter.

The power to the camera may be provided by a wired cable. In some cases, the cable wire may be in a wire bundle providing power to the camera as well as illumination elements or other circuitry at the distal tip of the catheter. The camera and/or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image and/or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure 1430 having a dimension matching a dimension of the miniaturized LED light source. As shown in the illustrated example, two cavities 1430 may be integrally formed with the catheter to receive two LED light sources. For instance, the outer diameter of the distal tip may be around 4 to 4.4 millimeters (mm) and diameter of the working channel of the catheter may be around 2 mm such that two LED light sources may be embedded at the distal end. The outer diameter can be in any range smaller than 4 mm or greater than 4.4 mm, and the diameter of the working channel can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiment, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover 1431 may be placed at the forwarding end face of the distal tip providing precise positioning of the LEDs as well as sufficient room for the glue. The cover 1431 may be composed of transparent material matching the refractive index of the glue so that the illumination light may not be obstructed.

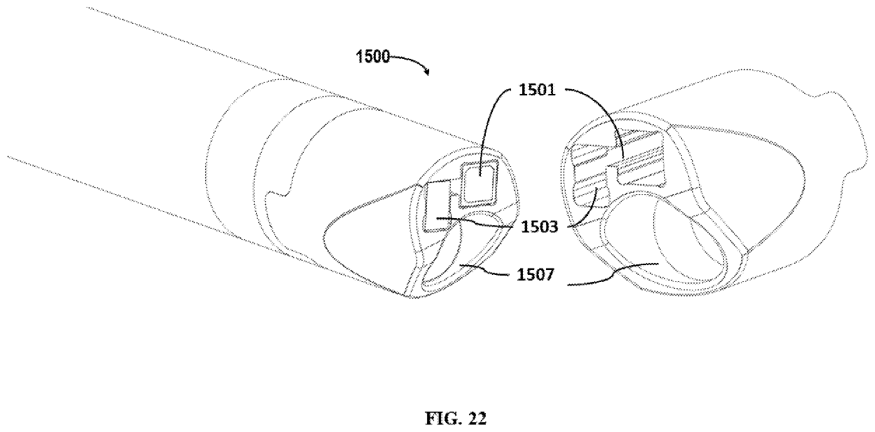

It should be noted that the illustrated distal end design is for illustration purpose only. There can be other suitable design for integrating the one or more components into the distal tip. FIG. 22 shows another example of a distal portion 1500 of the catheter with integrated imaging device and the illumination device. As shown in the example 1500, the distal tip may have a structure 1501 to receive the camera, a structure 1503 to receive an illumination device and/or the location sensor. The camera may be embedded into a cavity 1501 at the distal tip of the catheter. The cavity 1501 may be integrally formed with the distal portion of the cavity and may have a dimension matching a length/width of the camera such that the camera may not move relative to the catheter. The camera may be adjacent to the working channel 1507 of the catheter to provide near field view of the tissue or the organs. In some cases, the attitude or orientation of the imaging device may be controlled by controlling a rotational movement (e.g., roll) of the catheter. As shown in the illustrated example 1500, a cavity 1503 may be integrally formed with the catheter to receive an LED light source.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

The one or more processors may be in communication with the endoscope. The one or more processor may be located remotely from the endoscope system or onboard of the endoscope system (e.g., located at the handle, located at the user device for controlling the endoscope).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for estimating shape of an elongated member of an articulable flexible endoscope, the method comprising:
(a) receiving sensor data acquired by a series of sensor pairs located at multiple points along a length of the elongated member, wherein the series of sensor pairs are mounted to a structure movable relative to the elongated member;
(b) calculating orientations of the elongated member at the multiple points; and
(c) estimating a shape of the elongated member based at least in part on the orientations.

2. The method of claim 1, wherein each of the series of the sensor pairs comprise an accelerometer and a magnetometer.

3. The method of claim 2, wherein the structure is actuated to have a translational motion relative to the elongated member to increase a density of the multiple points.

4. The method of claim 3, wherein an impact of the translational motion is corrected from the orientations.

5. The method of claim 4, wherein the impact of the translation motion comprises an acceleration of the translational motion.

6. The method of claim 3, wherein the structures is actuated to move at a constant velocity.

7. The method of claim 3, wherein the structure is a slidable cable and wherein the series of sensor pairs are affixed to the slidable cable.

8. The method of claim 7, wherein the slidable cable is driven by an actuator located at an instrument driving mechanism removably coupled to the articulable flexible endoscope.

9. The method of claim 1, wherein the shape of the elongated member is estimated using linear approximation, circle section approximation, or cubic spline approximation.

10. The method of claim 9, wherein the shape of the elongated member is estimated using a combination of at least two of linear approximation, circle section approximation, or cubic spline approximation based at least in part on a spacing of the series of sensor pairs.

11. The method of claim 1, wherein the shape of the elongated member is estimated using a machine learning algorithm trained model.

12. The method of claim 11, wherein the model is automatically updated based at least in part on feedback data.

13. The method of claim 1, further comprising calibrating the series of sensor pairs based on a first known shape of the elongated member and a first corresponding series of orientations, and a second known shape of the elongated member and a second corresponding series of orientations.

14. The method of claim 13, wherein the first known shape is a straight configuration and wherein the second shape is an arc shape.

15. The method of claim 13, further comprising storing a calibration data in a memory device incorporated into the elongated member.

16. A non-transitory computer-readable storage medium including instructions that, when executed by one or more processors, cause the one or more processors to perform operations comprising:
(a) receiving sensor data acquired by a series of sensor pairs located at multiple points along a length of an elongated member of an articulatable flexible endoscope, wherein the series of sensor pairs are mounted to a structure movable relative to the elongated member;
(b) calculating orientations of the elongated member at the multiple points; and
(c) estimating a shape of the elongated member based at least in part on the orientations.

17. The non-transitory computer-readable storage medium of claim 15, wherein each of the series of the sensor pairs comprise an accelerometer and a magnetometer.

18. The non-transitory computer-readable storage medium of claim 17, wherein the structure is actuated to have a translational motion relative to the elongated member to increase a density of the multiple points.

19. The non-transitory computer-readable storage medium of claim 18, wherein an impact of the translational motion is corrected from the orientations.

20. The non-transitory computer-readable storage medium of claim 19, wherein the impact of the translation motion comprises an acceleration of the translational motion.
